(19) United States
(12) Patent Application Publication
(10) Pub. No.: US 2017/0246144 A1
(43) Pub. Date: Aug. 31, 2017

(54) METHOD OF PREVENTING OR TREATING SPINOCEREBELLAR ATAXIA BY ADMINISTRATING SILIBININ

(71) Applicant: National Taiwan Normal University, Taipei City (TW)

(72) Inventor: Jung-Yaw Lin, Taipei City (TW)

(21) Appl. No.: 15/441,473

(22) Filed: Feb. 24, 2017

(30) Foreign Application Priority Data
Feb. 25, 2016 (TW) 105105631

(51) Int. Cl. A61K 31/357; A61K 9/00

(52) U.S. Cl. CPC A61K 31/357; A61K 9/0019

(57) ABSTRACT

The present disclosure provides a use of silibinin for manufacturing a pharmaceutical composition for preventing or treating spinocerebellar ataxia.

### REFERENCE TO RELATED APPLICATION

This application claims foreign priority under 35 U.S.C. §119(a) to Patent Application No. 105105631, filed on Feb. 25, 2016, in the Intellectual Property Office of Ministry of Economic Affairs, Republic of China (Taiwan, R.O.C.), the entire content of which patent application is incorporated herein by reference.

### BACKGROUND OF THE INVENTION

1. Technical Field

The present disclosure relates to a use of silibinin for manufacturing a pharmaceutical composition, and more particularly, to a use of silibinin for manufacturing a pharmaceutical composition for the prevention or treatment of spinocerebellar ataxia (SCA).

2. Description of Related Art

SCA, also known as spinocerebellar atrophy or spinocerebellar movement disorder, is a common name of a collection of several progressive neurodegenerative disorders with similar symptoms. SCAs are inherited disorders with autosomal-dominant patterns through the involvement of various genes. The symptoms of SCAs are the gradual loss of balance and movement due to the damages of neurons in spinal cord and cerebellum, which causes the dysfunction of signaling transductions and signal connections (Ref. 1-3).

In recent years, 35 different types of SCA have been identified, which can be categorized into several groups according to distinct gene mutations. The first group includes SCA1, SCA2, SCA3, SCA6, SCA7 and SCA17. The abnormity is caused by the expanded repeats of CAG sequence within specific genes. Since CAG sequence encodes glutamine (Q), the proteins which are translated from the abnormal genes with CAG repeats contain long chain of glutamines (Ref. 4). This kind of proteins cannot fold properly and form protein aggregations (Ref. 5-6), which may lead to cell toxicity and cell death (Ref. 7-8). The second group includes SCAB, SCA10 and SCA 12. The abnormity is caused by the repeated expansion of non-translational regions contained nitrogenous bases (CTG-, ATTCT- and CAG-) within the derived variations of specific genes (KLHLIAS, ATXN10 and PPP2R2B) (Ref. 9-11). The third group includes SCA5, SCA14 and SCA27. The abnormity is caused by several mutations of specific genes (SPTBN2, PRKCG and FGF14), including deletion, non-sense, and translocation mutations (Ref. 12-14), wherein SCA17 is caused by an N-terminal aberrant poly-glutamine (polyQ) expansion in TATA-binding protein (TBP) (Ref. 15-17).

The polyQ chain of the normal TBP contains approximately 25-42 glutamines which are encoded by the repeats of CAG/CAA trinucleotides. The mutated TBP contains expanded polyQ chains (more than 42 glutamines) (Ref. 18-20). The symptoms of SCA17 include movement ataxia, progressive dysfunction of movement, and the degeneration of cerebellar Purkinje cells (Ref. 1, 20-21).

The pathogenesis mechanism of SCA17 remains unclear. Some investigations indicate that the de-regulation of calcium ion is involved in the pathogenesis of SCA17 transgenic mice (Ref. 22). The mutated TBP in mouse brain may contribute to the impairment of the homeostasis of calcium ion which is accumulated extracellularly in excess (Ref. 1, 23, 21). In addition, the protein aggregation induced by mutated TBP is associated with mitochondria dysfunction and caspase-dependent apoptosis (Ref. 24-25).

In relevant studies, it was suggested that polyQ expanded proteins tend to aggregate in forming nuclear or cytoplasmic inclusion bodies, which lead to neuro-degenerative disorders, such as Huntington's disease or SCAs (Ref. 26, 27). Prior studies also indicate that several SCAs are associated with the presence of protein aggregation and apoptosis in a specific region of the brain (Ref. 23). In 2001, the expansion of CAG fragment within TBP gene was identified and subsequent studies indicate that the expansion of polyQ can cause aggregation and intra-nuclear inclusion bodies (Ref. 17, 28). In particular, mutated TBP is also found in protein aggregations in other neurodegenerative disorders (Ref, 29, 26). In addition, some studies indicate that the aggregations of expanded TBP cause the release of cytochrome C from cytoplasm and induce cell death (Ref. 30-31).

The extracellular accumulation of glutamate excessively activates glutamate-related receptors, such as kainate acid (KA) receptor, N-methyl-d-aspartate (NMDA) receptor, α-amino-3-hydroxy-5-methylisoxazole-4-propionic acid (AMPA) receptor, and glutamate metabotropic receptor. The activation of these receptors opens up the receptor channels, which leads to the imbalance of calcium (Ref. 32-34). Several studies indicate that high concentration of glutamate is a potent neurotoxin which can destroy neurons through apoptosis (Ref. 32, 35). The excessive calcium inflow causes the increases of the reactive oxygen species (ROS) and leads to the dysfunction of mitochondria (Ref. 36-37).

Glutamatergic excitotoxicity is associated with the up-regulation of pro-apoptosis protein, Bax, and the down-regulation of anti-apoptosis protein, Bcl-2. Previous studies indicate that neuronal apoptosis in response to the stimulation of various glutamate receptors is mediated by the signaling of caspase family (Ref. 38-40). In addition, the protein expression levels of calpain-2 and calpain-specific α-spectrin breakdown product (SBDP) are both Ca²⁺ dependent, which are elevated to aggravate the damages in glutamate-induced cell injury (Ref. 41-42).

Oxidative stress induced by ROS or free radicals plays an important role in the pathogenesis of neurodegenerative disorders. When the accumulation of calcium by mitochondria reaches the critical level, it perturbs functional activity and causes permeability transition in the inner mitochondrial membrane. Eventually it leads to mitochondria damages (Ref. 43) and neuronal death (Ref. 44-45). Increased mitochondrial calcium loading induced by glutamate is positively associated with ROS generated by mitochondria (Ref. 46). If mitochondria are affected or damaged, the overly generated ROS would contribute oxidative stress and cell toxicity (Ref. 47). Glutamatergic excitotoxicity has been shown to induce oxidative stress which is associated with the dysfunction of mitochondria in neurodegenerative disorders (Ref. 48).

Currently there is no effective method in treating SCA. The current treatment for SCA is limited to the relief of symptoms and improving the life quality of patients, such as physical therapy by exercising or medication administration for specific symptoms, for example, the application of effective medication or supplemental therapy for fatigue, insomnia, tremor, stiff, depression, pain and infection. However, these drugs have known side effects. The impacts of the side effects of these drugs could be more severe to the patients with relevant movement disorders. Therefore, currently it is in need for a drug and treatment method to relieve and treat SCA without having severe side effects.

### SUMMARY OF THE INVENTION

In relevant to the aforementioned issues, the present disclosure provides a use of silibinin for manufacturing a pharmaceutical composition for prevention or treatment of SCA.

According to one embodiment of the present disclosure, said SCA relates to aberrant accumulation of polyQ proteins, e.g., the first group of SCA including SCA1, SCA2, SCA3, SCA6, SCAT and SCA17. According to one embodiment of the present disclosure, said SCA is SCA17.

According to one embodiment of the present disclosure, a concentration of the silibinin in the pharmaceutical composition is within a range from 5 μM to 30 μM, preferably from 5 μM to 25 μM, more preferably from 10 μM to 20 μM. The effective amount of silibinin is in a range from 0.125 mg to 9 mg, preferably from 1.5 mg to 7.5 mg, more preferably from 3 mg to 6 mg, per kilogram of a body weight of said subject.

In another embodiment, the present disclosure is to provide a use of silibinin for manufacturing a pharmaceutical composition for prevention or treatment of damage of neuronal cells. In one embodiment, the neuron damage is caused by glutamatergic excitotoxicity and/or apoptosis induced by glutamate. According to one embodiment of the present disclosure, the apoptosis induced by glutamate is mediated by mitochondria.

According to an embodiment of the present disclosure, the pharmaceutical composition of the present disclosure is to prevent or treat damage of neuronal cells by inhibiting an apoptosis pathway. According to an embodiment of the present disclosure, the apoptosis pathway is a calcium dependent apoptosis pathway, a mitochondria dependent apoptosis pathway and/or a caspase dependent apoptosis pathway.

In another embodiment of the present disclosure, the present disclosure is to provide a use of silibinin for manufacturing a pharmaceutical composition for prevention or treatment of damage of neuronal cells. In an embodiment, the neuron damage relates to aberrant accumulation of polyQ proteins. In an embodiment, the pharmaceutical composition is applied to the subject in need thereof, and an effective amount of the silibinin for treating the subject is in a range from 0.125 mg to 9 mg per kilogram of a body weight of the subject. According to an embodiment of the present disclosure, the neuronal damage related to the aberrant accumulation of polyQ proteins is the SCA17.

The silibinin provided by the present disclosure can effectively treat the damages of neuronal cells and can inhibit the apoptosis pathway caused by a calcium dependent apoptosis pathway, a mitochondria dependent apoptosis pathway and/or a caspase dependent apoptosis pathway. It can also reduce the protein aggregation in neuronal cells to effectively prevent or treat SCA and/or the damages of neuronal cells.

### BRIEF DESCRIPTIONS OF THE DRAWINGS

FIGS. 1A and 1B show the effects of silibinin (SB) in reducing the death of SH-SY5Y cell induced by mono-sodium glutamate (MSG). FIG. 1A shows the cell viabilities of SH-SY5Y cell treated with various concentrations of SB after 24 hours. FIG. 1B shows the cell viabilities of SH-SY5Y cell treated with various concentrations of SB or 10 μM NMDA receptor antagonist, MK801, and 100 mM MSG after 24 hours. Data were the mean values of three independent experiments. Ctrl: control group.

FIG. 2 shows the effects of SB in the apoptosis induced by 100 mM MSG. 1×10⁶ SH-SY5Y cells were treated with 100 mM MSG and 15 μM SB or 10 μM MK801 (MK) for 24 hours. Cells were stained with Annexin-V/PI and the apoptosis was measured with a flow cytometry.

FIG. 3 shows the effects of SB on the levels of calpain-2 and SBDPs induced by MSG. The levels of calpain-2 and SBDPs were measured by western blotting in 1×10⁶ SH-SY5Y cells, wherein the control group (Ctrl) was untreated SH-SY5Y cells or the cells were treated with 100 mM MSG, 100 mM MSG+15 μM SB or 100 mM MSG+10 μM MK801 (MK) for 6 hours. Actin was used as a loading control.

FIG. 4 shows the effects of SB on the levels of Bax and Bcl-2 in SH-SY5Y cells after the treatment of MSG. The levels of Bax and Bcl-2 in 1×10⁶ SH-SY5Y cells were measured by western blotting, wherein the control group (Ctrl) was untreated SH-SY5Y cells or the cells were treated with 100 mM MSG, 100 mM MSG+15 μM SB or 100 mM MSG+10 μM MK801 (MK) for 6 hours. Actin was used as a loading control.

FIGS. 5A-5D show the effects of SB on the levels of cleaved caspase-9, cleaved caspase-3, and cleaved poly(ADP-ribose) polymerase (PARP) in SH-SY5Y cells treated with MSG FIG. 5A: The levels of Bax and Bcl-2 in 1×10⁶ SH-SY5Y cells were measured by western blotting, wherein the control group (Ctrl) was untreated SH-SY5Y cells or the cells were treated with 100 mM MSG, 100 mM MSG+15 μM SB or 100 mM MSG+10 μM MK801 (MK) for 24 hours. Actin was used as a loading control. FIGS. 5B-5D show the quantification results measured by western blotting. Data were the mean values of three independent experiments.

FIGS. 6A and 6B show that SB can reduce the ROS production induced by MSG in SH-SY5Y cells. FIG. 6A: The ROS productions in 1×10⁶ SH-SY5Y cells were measured by chemiluminescence (CL) analysis and luminol measurement, wherein the control group (Ctrl) was untreated SH-SY5Y cells or the cells were treated with 100 mM MSG, 100 mM MSG+15 μM SB or 100 mM MSG+10 μM MK801 (MK) for 24 hours. FIG. 6B shows the quantification results measured by CL analysis.

FIGS. 7A and 7B show the effects of SB on mitochondria membrane potential (MMP) in SH-SY5Y cells treated with MSG measured by JC-1 staining with a flow cytometry, wherein the control group (Ctrl) was untreated SH-SY5Y cells or the cells were treated with 100 mM MSG, 100 mM MSG+15 μM SB, 100 mM MSG+10 μM MK801 (MK) or 5 μM CCCP (disruptor of electron transport chain) for 12 hours. FIG. 7B shows the ratio of JC-1 oligomer/monomer. The results were shown as mean±SEM, n=3, *p<0.05, comparing to the MSG treatment group.

FIGS. 8A-8C show the effects of SB on improving the viability of nTBP/Q₇₉-EGFP cells and inhibiting the expression levels of cleaved caspase-9, cleaved caspase-3, and cleaved PARP in nTBP/Q₇₉-EGFP cells induced by doxycycline (Dox). FIG. 8A: The viabilities of 1.0×10⁶ nTBP/Q₃₆-EGFP cells and nTBP/Q₇₉-EGFP cells which were pretreated with 15 μM SB for 1 hour followed by the induction of Dox for 1, 3, and 5 days were measured by MTT assay. FIG. 8B: 1.0×10⁶ nTBP/Q₃₆-EGFP cells and nTBP/Q₇₉-EGFP cells were pretreated with 15 μM SB for 1 hour followed by the induction of Dox for 5 days. The levels of cleaved caspase-9, cleaved caspase-3, and cleaved PARP were detected by western blotting. Actin was used as a loading control. FIG. 8C shows the quantification results of the western blotting. The results were shown as mean±SD, n=3, *p<0.05, comparing to the Dox group.

FIGS. 9A-9C show the effects of SB on protein aggregation in nTBP/Q₇₉-EGFP cells after the induction of Dox, which were analyzed by western blotting and dot blot assays. 1.0×10⁶ nTBP/Q₃₆-EGFP cells or nTBP/Q₇₉-EGFP cells were pretreated with 15 μM SB for 1 hour followed by the treatment of 10 μg/mL Dox. Cell lysates were analyzed using western blotting and dot blot assays with anti-TBP (N12) antibody. FIG. 9B shows the quantification results of the intensities of TBP (N12) protein in nTBP/Q₇₉-EGFP cells. The values represent means±SD. Data were the mean values of three independent experiments.

FIGS. 10A-10F show the effects of SB on the body weight and motor performance in SCA17 transgenic mice. Mice were grouped into wild type-saline (WT-saline), transgenic-saline (TG-saline), and transgenic-SB treatment (TG-SB) groups. Mice were injected intraperitoneally with saline or SB (4.5 mg/kg) at 8-week old once every 2 days. FIG. 10A shows the body weights of the mice from 8-week old to 20-week old. FIG. 10B shows the performance of motor coordination of the mice determined by rotarod test. FIG. 10C shows the footprint patterns of the mice at 20-week old. FIG. 10D shows the overlapping of the footprints of front-limb and hind-limb. FIG. 10E and FIG. 10F show respectively the stride length of front-limb/hind-limb at the right-side and the left-side. The values represent means±SD, n=6. *p<0.05, comparing to the TG-saline group.

FIGS. 11A and 11B show the effects of SB on the levels of TBP (N12) and cleaved caspase-3 in SCA17 transgenic mice. Mice were grouped into wild type-saline (WT-saline), transgenic-saline (TG-saline), and transgenic-SB treatment (TG-SB) groups. Mice were injected intraperitoneally with saline or SB (4.5 mg/kg) at 8-week old once every 2 days. FIG. 11A: The extent of TBP aggregation (detected with TBP (N12) antibody) and the levels of cleaved caspase-3 in the cerebella of mice were measured by western blotting at 20-week old. Actin was used as a loading control. FIG. 11 B shows the quantification results of the intensities of TBP (N12) and the level of cleaved caspase-3 protein. The results were shown as mean±SEM, n=6, *p<0.05, comparing to the TG-saline group.

### DETAILED DESCRIPTIONS OF THE INVENTION

The present disclosure is described by using the following embodiments, so as to enable a person skilled in the art to conceive the other advantages and effects of the present disclosure from the disclosure of the present specification. However, the examples in the present disclosure are not used for limiting the scope of the present application. Any one skilled in the art can alter or modify the present disclosure in any way, without departing from the spirit and scope thereof. Therefore, the scope of the present disclosure should be accorded with the definitions in the appended claims.

The present disclosure provides a use of silibinin for manufacturing a pharmaceutical composition for the prevention or treatment of SCA, wherein the pharmaceutical composition is applied to the subject in needs of the treatment.

According to an embodiment of the present disclosure, said SCA is related to the aberrant accumulation of polyQ proteins, preferably said SCA is the seventeenth type spinocerebellar ataxia.

According to an embodiment of the present disclosure, silibinin can reduce the aberrant accumulation of polyQ proteins in the subject

According to the embodiments of the present disclosure, a concentration of the silibinin in the pharmaceutical composition is within a range from 5 μM to 30 μM, preferably at the concentration of 15 μM.

For the pharmaceutical composition provided by the present disclosure, the effective amount to apply to the subject can be variable depending on the condition for adjusting the dosage of medicament known in the field, such as condition of the subject (including species, gender and age), severity of the diseases, other diseases, and other acceptable treatment. According to an embodiment of the present disclosure, the effective amount of silibinin is in a range from 0.125 mg to 9 mg per kilogram of body weight in treating said subject.

Said subject may include, but not limited to, for example, mice, rat, hamster, guinea pig, mink, rabbit, dog, primate, pig, cow, sheep and so on. When the treated subjects are different species, the effective amount of the pharmaceutical composition provided by the present disclosure can be adjusted as needed according to the adjusting methods known in the technical field, for example, the conversion methods of dosage amount for different species in clinical trials as disclosed in the publication of FDA, Guidance for Industry: Estimating the Maximum Safe Starting Dose in Initial Clinical Trials for Therapeutics in Adult Healthy Volunteers (2005). This publication is herein incorporated by reference.

According to an embodiment of the present disclosure, the effective amount of the pharmaceutical composition for applying to the subject may be based on the concentration range of silibinin (from 5 μM to 30 μM) as described in the previous embodiment, which can be converted to from 1.5 mg to 9 mg of silibinin per kilogram of body weight. In one embodiment, the effective amount is in a range from 3 mg to 7.5 mg of silibinin per kilogram of body weight or 4.5 mg of silibinin per kilogram of body weight. In the present embodiment, the subject may be mammal, such as mice.

According to another embodiment of the present disclosure, the effective amount of silibinin comprised in the pharmaceutical composition for applying to the subject, preferably, may be in a range from 0.125 mg to 0.75 mg per kilogram of body weight, or from 0.25 mg to 0.625 mg per kilogram of body weight, or 0.375 mg per kilogram of body weight. In the present embodiment, the subject may be mammal, such as human.

According to an embodiment of the present disclosure, the frequency of the administration of the pharmaceutical composition is depending on the subject as needed, for example, three times a day, twice a day, once a day, or once in two days.

According to an embodiment of the present disclosure, the administration methods of the pharmaceutical composition may be oral, parenteral, intravenous, or injected.

The present disclosure provides a use of silibinin for manufacturing a pharmaceutical composition for the prevention or treatment of the damages of neuronal cells.

According to an embodiment of the present disclosure, the damages of neuronal cells are caused by glutamatergic excitotoxicity and/or apoptosis induced by glutamate. According to another embodiment of the present disclosure, the apoptosis induced by glutamate is mediated by mitochondria.

In another aspect of the present disclosure, the present disclosure provides a use of silibinin for manufacturing a pharmaceutical composition for the prevention or treatment of the damages of neuronal cells by inhibiting the apoptosis pathway of the neuronal cells.

According to an embodiment of the present disclosure, the apoptosis pathway is a calcium dependent apoptosis pathway, a mitochondria dependent apoptosis pathway and/or a caspase dependent apoptosis pathway.

According to an embodiment of the present disclosure, silibinin can inhibit the expression of calpain-2 and/or SBDP.

According to an embodiment of the present disclosure, silibinin can inhibit the expression of Bax and increase the expression of Bcl-2.

According to an embodiment of the present disclosure, silibinin can inhibit the expression of the proteins which are related to caspase, for example, silibinin can inhibit the expression of cleaved-caspase-9, cleaved-caspase-3, and cleaved-PARP.

According to an embodiment of the present disclosure, silibinin can reduce the production of intracellular reactive oxygen species induced by glutamate.

According to an embodiment of the present disclosure, silibinin can reduce the loss of mitochondria membrane potential and/or the protein aggregation in neuronal cells induced by glutamate.

In another aspect of the present disclosure, the present disclosure provides a use of silibinin for manufacturing a pharmaceutical composition for the prevention or treatment of the damages of neuron related to the aberrant accumulation of polyQ proteins.

The pharmaceutical composition is administrated to the subject that has the need of the pharmaceutical composition. When the pharmaceutical composition is administrated to the subject, the effective amount may be in a range from 0.125 mg to 9 mg of silibinin per kilogram of the body weight.

Said subject may include, but not limited to, for example, mice, rat, hamster, guinea pig, mink, rabbit, dog, primate, pig, cow, sheep and so on. When the treated subjects are different species, the effective amount of the pharmaceutical composition provided by the present disclosure can be adjusted as needed according to the adjusting methods known in the technical field, for example the conversion methods of dosage amount for different species in clinical trials as disclosed in the publication of FDA, Guidance for Industry: Estimating the Maximum Safe Starting Dose in Initial Clinical Trials for Therapeutics in Adult Healthy Volunteers (2005). This publication is herein incorporated by reference.

According to an embodiment of the present disclosure, the effective amount of the pharmaceutical composition for applying to the subject may be based on the concentration range of silibinin (from 5 μM to 30 μM) as described in the previous embodiment, which can be converted to from 1.5 mg to 9 mg of silibinin per kilogram of body weight. In one embodiment, the effective amount is in a range from 3 mg to 7.5 mg of silibinin per kilogram of body weight or 4.5 mg of silibinin per kilogram of body weight. In the present embodiment, the subject may be mammal, such as mice.

According to another embodiment of the present disclosure, the effective amount of silibinin comprised in the pharmaceutical composition for applying to the subject, preferably, may be in a range from 0.125 mg to 0.75 mg per kilogram of body weight, or from 0.25 mg to 0.625 mg per kilogram of body weight, or 0.375 mg per kilogram of body weight. In the present embodiment, the subject may be mammal, such as human.

According to an embodiment of the present disclosure, the neuronal damage related to the aberrant accumulation of polyQ proteins is the seventeenth type spinocerebellar ataxia.

The effects of the present disclosure are further illustrated by the following specific embodiments, which are not intended to limit the scope of the present disclosure.

### EXAMPLES

### Materials:

Dulbecco's Modified Eagle Medium with nutrient mixture F-12 (DMEM/F12), 0.5% Trypsin-EDTA, penicillin/streptomycin (P/S), and Fluo-4 AM were obtained from Invitrogen Corporation. Fetal bovine serum (FBS) was from Falcon. The primary antibodies against calpain-2, Bax, cleaved PARP, cleaved caspase-9, and cleaved caspase-3 were obtained from Cell Signaling Technology. Cytochrome C, Bcl-2, TBP (1C2) and TBP (N12) were purchased from Santa Cruz Technology. Actin and SBDPs were purchased from Millipore Corporation. Secondary antibodies of horseradish peroxidase (HRP)-conjugated goat anti-mouse antibody and goat anti-rabbit antibody were obtained from Millipore Corporation. 3-(4,5-cimethylthiazol-2-yl)-2,5-diphenyl tetrazolium bromide (MTT) and retinoic acid (RA) were purchased from Sigma-Aldrich. Annexin V-FITC assay kit was purchased from Invitrogen Corporation. Protease inhibitors cocktail was obtained from Roche Applied Science. Pure compounds used in the present disclosure were purchased from Sigma-Aldrich.

### Cell Culture

Human neuroblastoma SH-SY5Y cell line was obtained from American Type Culture Collection (ATCC). nTBP/Q₃₆-EGFP cells (poly-Q of TATA-binding protein contains 36 glutamines in SH-SY5Y cells) and nTBP/Q₇₉-EGFP cells (poly-Q of TATA-binding protein contains 79 glutamines in SH-SY5Y cells) were kindly supplied by Dr. Guey-Jen Lee-Chen, National Taiwan Normal University (NTNU). Cells were cultured in DMEM/F12 media supplemented with 10% FBS, 100 U/mL penicillin and 100 μg/mL streptomycin at 37° C. in a 5% CO₂ humidified incubator.

### Example 1: The Effects of Silibinin in Resisting Glutamatergic Excitotoxicity in SH-SY5Y Cells

In order to monitor the cyto-toxicity of silibinin and its ability in resisting glutamatergic excitotoxicity, MTT colorimetric assay was used to measure the cell viability rate of neuroblastoma SH-SY5Y cells which were pre-treated with silibinin and mono-sodium glutamate (MSG).

SH-SY5Y cells were plated in 96-well plates (2×10⁴ cells/well). After 24 hours, cells were pre-treated with various concentrations of silibinin or 10 μM MK801 (known NMDA receptor antagonist) for 1 hour followed by the treatment of 100 mM MSG for 24 hours.

After the treatment, 0.5 mg/mL MTT was added to the culture media, and the cells were incubated for 3 hours at 37° C. 100 μL lysis solution (10% sodium dodecyl sulfate (SDS) and 0.01N HCl) was added and the absorbance of the media was read at OD₅₇₀. The percentage of cell viability was calculated as follows:

Cell viability (%)=(OD₅₇₀ of experimental well/OD₅₇₀ of control well)×100%

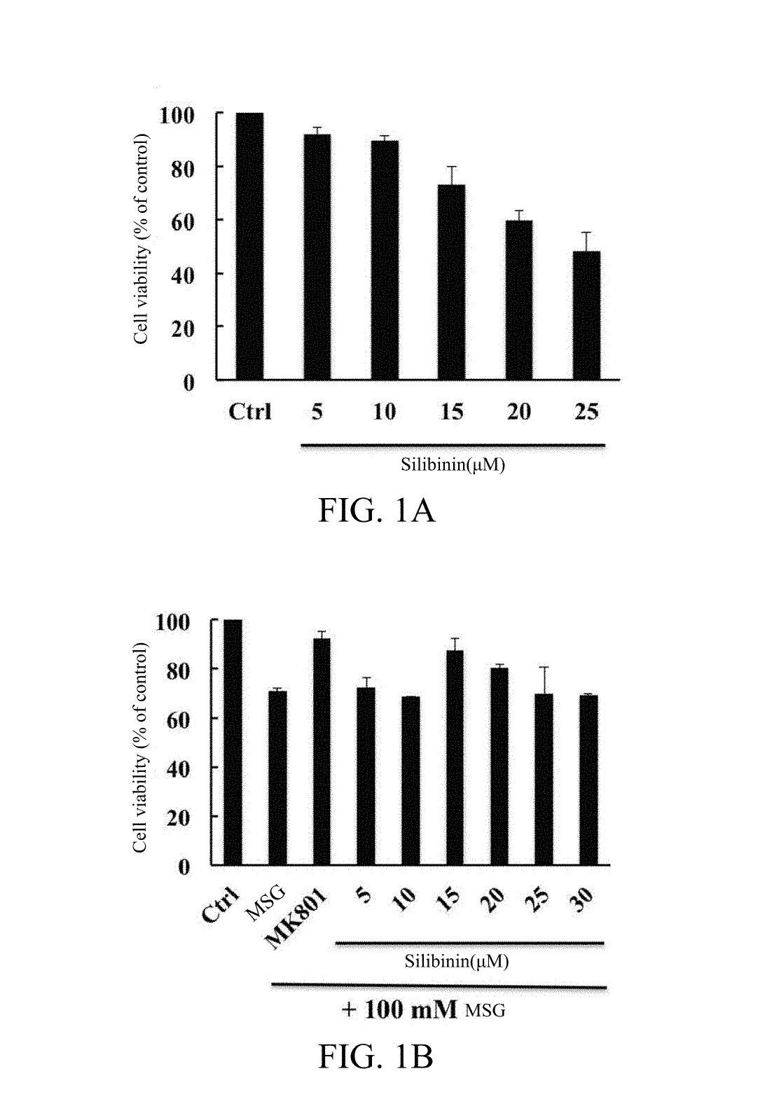

FIG. 1A shows that the cyto-toxicity of silibinin toward SH-SY5Y cells was dose-dependent with IC₅₀=30 μM.

FIG. 1B shows the viability of SH-SY5Y cells which were treated with MSG for 24 hours in the presence of 5, 10, 15, 20, 25 or 30 μM of silibinin. The results indicate that silibinin provided protective effects toward SH-SY5Y cells which were treated with MSG. Under the conditions of using 15-20 μM of silibinin or 10 μM MK801 respectively, the viability rates of SH-SY5Y cells which were exposed to 100 mM MSG for both conditions were equal. Therefore, silibinin demonstrates excellent inhibitory effects toward excitotoxicity induced by glutamate.

### Example 2: The Effects of Silibinin in Inhibiting the Apoptosis Mediated by Glutamate

In order to detect the effects of silibinin in inhibiting the apoptosis induced by glutamate in SH-SY5Y cells. The cells were measured by flow cytometry using Annexin V-FITC/propidium iodide (PI) double-labeling method.

SH-SY5Y cells were seeded in dish and were treated with 100 mM MSG in combination of 15 μM silibinin or 10 μM MK801 for 24 hours. The cells were then trypsinized and collected by centrifugation at 2,000 rpm for 3 mM followed by double-staining with Annexin V-FITC and PI. Stained cells were analyzed with a flow cytometry (FACS Sorter, Becton, Dickinson and Company). 10,000 cells were recorded in each experiment.

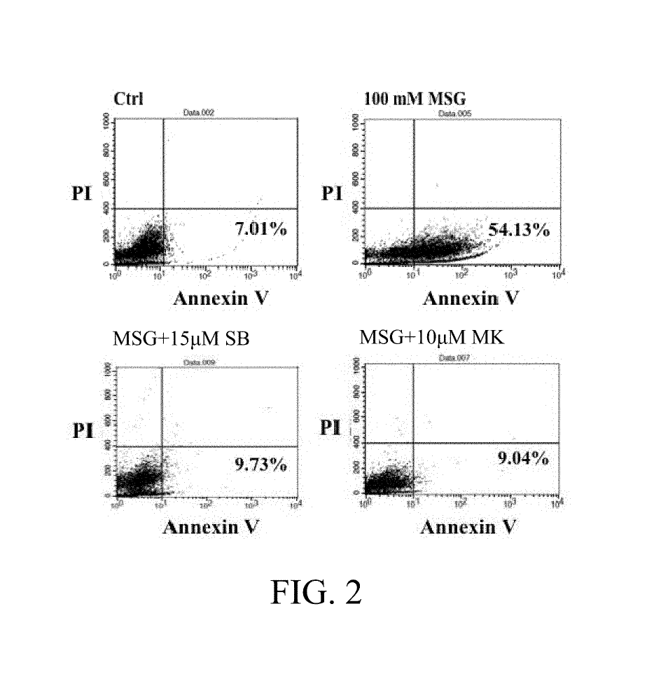

The results in FIG. 2 demonstrate that the treatment of 100 mM MSG for 24 hours induced 54.13% apoptotic cells, however, silibinin at 15 μM and MK801 at 10 μM caused 9.04% and 9.73% apoptosis, respectively. It indicates that silibinin effectively reduced apoptosis of SH-SY5Y cells induced by glutamate. In addition, the pretreatment of silibinin can protect cells from the apoptosis induced by glutamate.

### Example 3

In previous study, it has been indicated that glutamatergic excitotoxicity and the loss of mitochondria membrane potential are related to imbalance of calcium ion. When hippocampus neurons are treated with glutamate, the increases of calcium ion in cytosol trigger the activation of calpain. In addition, glutamatergic excitotoxicity induces the apoptosis which is mediated by mitochondria-dependent apoptosis pathway. Said mitochondria-dependent apoptosis pathway plays a very important role in neurodegenerative diseases. The cytochrome C released from inner-membrane of mitochondria will bind to Apaf-1 upon entering cytoplasm, and further activate caspase-9 and caspase-3. Further, the mitochondria-dependent apoptosis pathway is mediated by the members of Bcl-2 family, such as Bax and Bcl-2. It can affect the neuronal death mediated by calcium ion by regulating the permeability of mitochondria membrane. Therefore, the present embodiment further measures the effects of silibinin on the apoptosis pathway induced by glutamate.

### The Effects of Silibinin on the Expression of Calpain-2 and SBDP in Cells:

In present embodiment, western blotting was performed to study whether silibinin protects SH-SY5Y cell from apoptosis due to glutamatergic excitotoxicity via calcium-dependent apoptosis pathway.

Calpain-2, a thiol proteinase, is activated by the increases of intracellular free calcium ions and the reduction of Bcl-2 level. The level of SBDPs was elevated by calcium-induced calpain-2 in glutamate-induced cell death. Therefore, calpain-2 and SBDPs were used as indicators in the present embodiment.

SH-SY5Y cells were treated with silibinin at pre-determined concentrations or 10 μM MK801 for 1 hour followed by the treatment of 100 mM MSG. Western blotting was performed to analyze the levels of calpain-2 and SBDP.

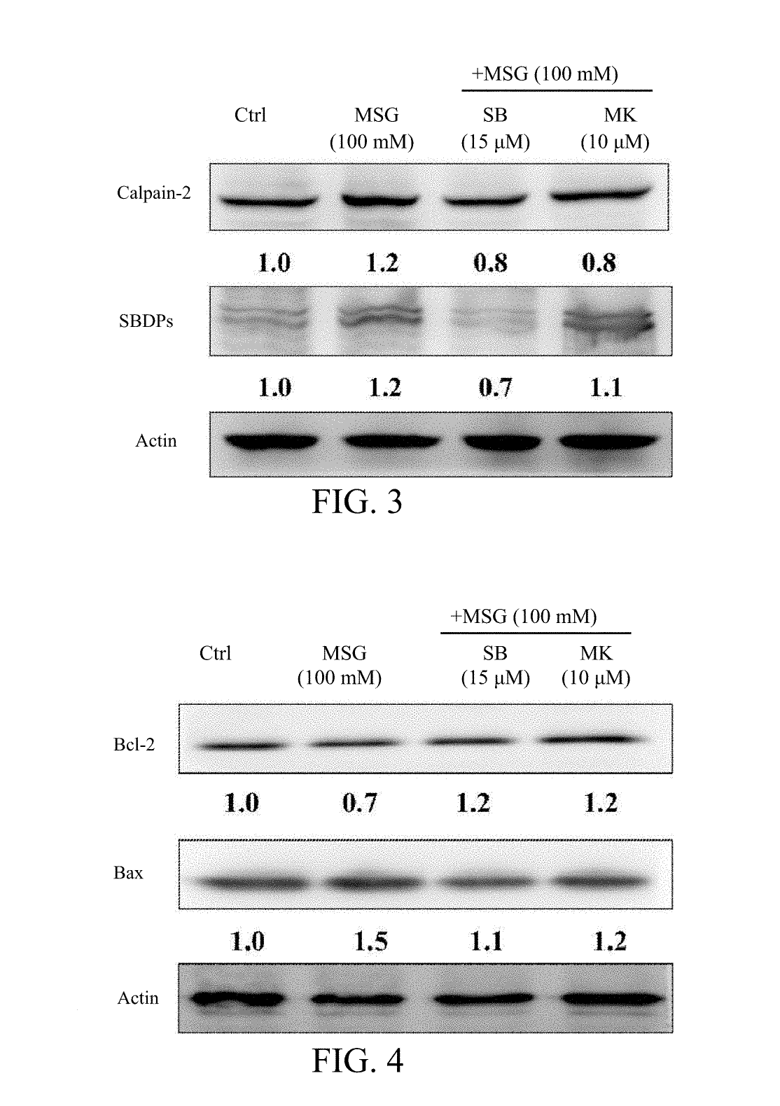

As shown in FIG. 3, both levels of calpain-2 and SBDP in SH-SY5Y cells increased 20% after the treatment of 100 mM MSG for 6 hours. When compared to the increased levels in the group treated only with 100 mM MSG, the levels of calpain-2 and SBDP reduced 200% and 250% respectively in the group treated with 100 mM MSG in combination of 15 μM silibinin. The results indicate that silibinin effectively protects SH-SY5Y cells treated with glutamate from apoptosis induced by calcium ion.

### The Effects of Silibinin on the Expression of Bax and Bcl-2 in Cells:

In present embodiment, western blotting was performed to study whether silibinin protects SH-SY5Y cell from apoptosis due to glutamatergic excitotoxicity via mitochondria-dependent apoptosis pathway.

It is known that the reduction of Bcl-2 level and the increases of Bax level are related to the increases of free calcium ion in cytosol. Bcl-2 is a cell-survival protein and Bax is a pro-apoptotic protein and they both mediate the releases of cytochrome C from mitochondria. Therefore, Bcl-2 and Bax are used as indicators in the present embodiment.

As shown in FIG. 4, the level of Bax increased 50% and the level of Bcl-2 reduced 30% in cells treated with 100 mM MSG for 6 hours. When compared to the detected levels in the group treated only with 100 mM MSG, the levels of Bax reduced 80% and the levels of Bcl-2 increased approximately 167% in the cells treated with 100 mM MSG in combination of 15 μM silibinin for 6 hours. In addition, the effect of 15 μM silibinin in increasing the expression level of Bcl-2 was equivalent to that of MK801. The effect of 15 μM silibinin in reducing the expression level of Bax was better than that of MK801. The results indicate that silibinin effectively protects SH-SY5Y cells treated with glutamate from mitochondria-dependent apoptosis.

### The Effects of Silibinin on Expressions of Caspase Family Proteins Mediated by Glutamate:

Caspase belongs to cysteine protease family which is typically closely related to apoptosis, wherein the activation of caspase-9, caspase-3 and ribozyme PARP is associated with delayed excitotoxicity damages. Therefore, western blotting was performed to analyze whether silibinin can protect SH-SY5Y cells from death via caspase-dependent apoptosis pathway in the present embodiment.

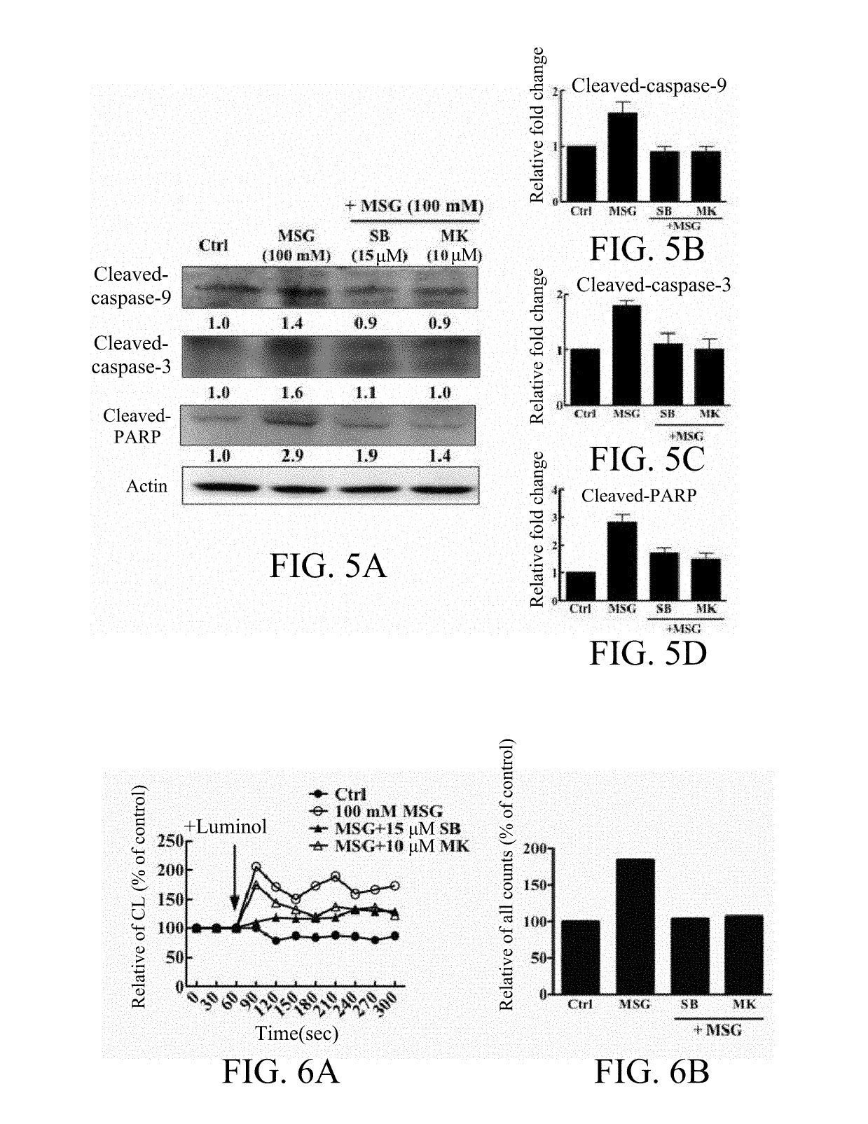

As shown in FIGS. 5A-5D, the levels of cleaved-caspase-9, cleaved-caspase-3 and cleaved-PARP increased 40%, 60% and 190% respectively in the cells treated with 100 mM MSG for 24 hours.

When compared to the increased levels in the group treated only with 100 mM MSG, the levels of cleaved-caspase-9, cleaved-caspase-3 and cleaved PARP reduced 125%, 83% and 52% respectively in the cells treated with 100 mM MSG in combination of 15 μM silibinin for 24 hours. The results indicate that silibinin can increase cell viability rate by inhibiting the expression of caspase family proteins, which are mediated by glutamate.

### Example 4: The Inhibition of Intracellular ROS by Silibinin

It is known that the cyto-toxicity induced by glutamate is associated with mitochondria dysfunction and the increased production of ROS in neuron. In this embodiment, chemiluminescence analysis was performed to measure the level of ROS in order to understand whether silibinin can inhibit the accumulation of ROS associated with glutamatergic excitotoxicity. In the chemiluminescence analysis, luminol was activated by oxidant to exhibit chemiluminescence. The emission spectrum was blue light which can be monitored by chemiluminescence detector.

SH-SY5Y cells were seeded in dish (1×10⁶ cells/plate) for 24 hours, and then were pre-treated with 15 μM silibinin or 10 μM MK-801 for 1 hour followed by the treatment of 100 mM glutamate for 24 hours. The cells were then washed and suspended in 60 μL RIPA on ice bath followed by stirring and centrifugation at 13,000 rpm for 20 mM at 4° C. The obtained protein sample (200 μL, 20 μg) was mixed with 0.5 mL of 0.2 mM luminol (Sigma). After 5 minutes, chemiluminescence analysis system (CLD-110, Tohoku Electronic Inc. Co. Japan) was performed to determine its chemiluminescence.

As shown in FIGS. 6A and 6B, the excitotoxicity induced by 100 mM MSG lead to the increases of intracellular ROS in approximately 80% without impacting the extracellular ROS. After the treatment of glutamate, when the cells were treated with 10 μM silibinin or 10 μM MK801 for 24 hours, there was 80% inhibition of intracellular ROS in the cells by maintaining extracellular ROS unchanged. When compared to MK801, silibinin can achieve the effects of inhibiting ROS more quickly. The results indicate that silibinin can reduce the production of intracellular ROS induced by glutamate.

### Example 5. Preventing the Reduction of Mitochondria Membrane Potential (MMP) by Silibinin

In the present embodiment, flow cytometry analysis was used to investigate whether silibinin can prevent the reduction of mitochondria membrane potential induced by glutamate in SH-SY5Y cells.

SH-SY5Y cells were seeded in 6 centimeter dish (1×10⁶ cells/plate) for 24 hours, and then were pre-treated with 15 μM silibinin or 10 μM MK-801 for 1 hour followed by the treatment of 100 mM MSG for 12 hours. 5 μM carbonyl cyanide m-chlorophenylhydrazone (CCCP) was added to the positive control group to induce the depolarization of mitochondria membrane potential.

The treated cells were washed, centrifuged and collected, followed by PBS wash and JC-1 dye staining for 30 minutes. The stained cells were re-suspended using phosphate buffer solution and analyzed by a flow cytometry to obtain a 2-dimensional scatter plot. 10,000 cells were recorded for each experiment.

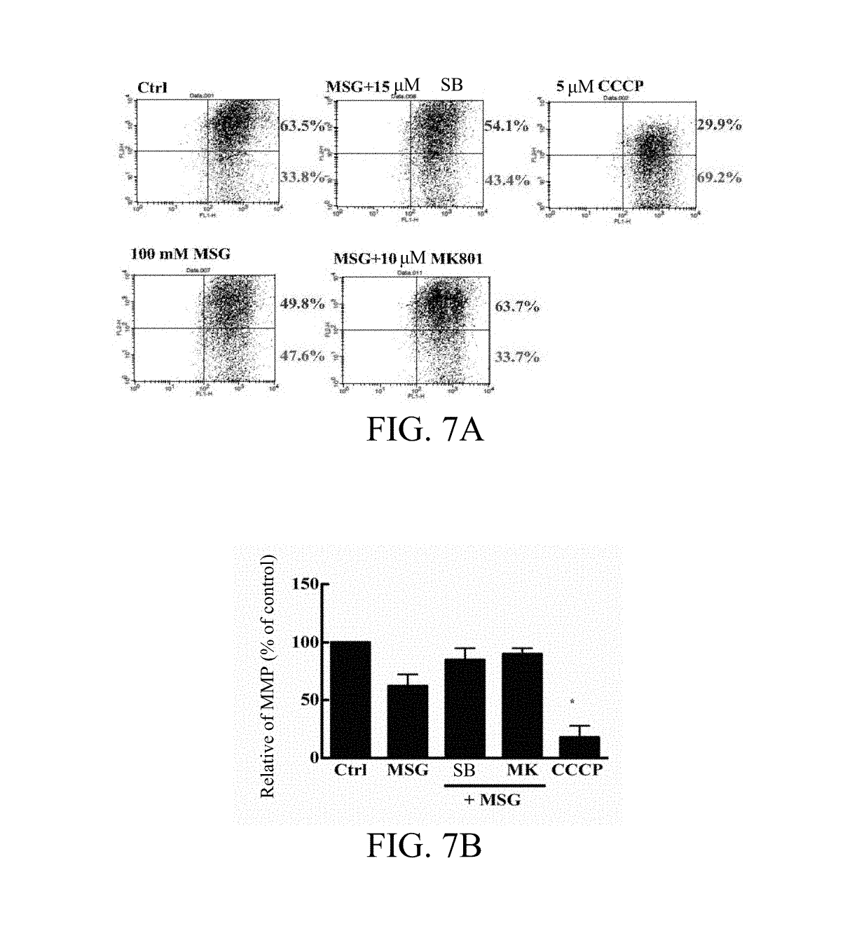

As shown in FIGS. 7A and 7B, when compared to the control group (Ctrl), the cells which were treated with 100 mM MSG for 24 hours only showed 62+10% MMP. However, when compared to Ctrl, the cells which were treated with 15 μM silibinin+100 mM MSG and treated with 10 μM MK801+100 mM MSG showed 85+13% MMP and 90±5% MMP respectively. For the cells treated with CCCP (disruptor of electron transport chain), only 18±10% MMP were retained. It is known that the over-loading of calcium ion in neuron cells can trigger the loss of mitochondria membrane potential and the production of ROS which lead to apoptosis. It demonstrates that silibinin can maintain stable MMP by effectively reducing the loss of MMP induced by glutamate.

### Example 6. The Effects of Silibinin in Cells Containing Poly-Glutamine

Viability of nTBP/Q₇₉-EGFP Cells Induced by Dox

In order to further measure the effects of silibinin in treating SCA, inducible nTBP/Q₃₆-EGFP cells and nTBP/Q₇₉-EGFP cells were used.

nTBP/Q₃₆-EGFP cells and nTBP/Q₇₉-EGFP cells were seeded in 96 well plates (2×10⁴ cells/well). After 24 hours, these cells were pre-treated with different concentrations of silibinin for one day followed by the treatment of 10 μg/mL Dox and retinoic acid for 1, 3, and 5 days to induce the expression of nTBP/Q₃₆-EGFP and nTBP/Q₇₉-EGFP in cells. The control group was not treated by silibinin.

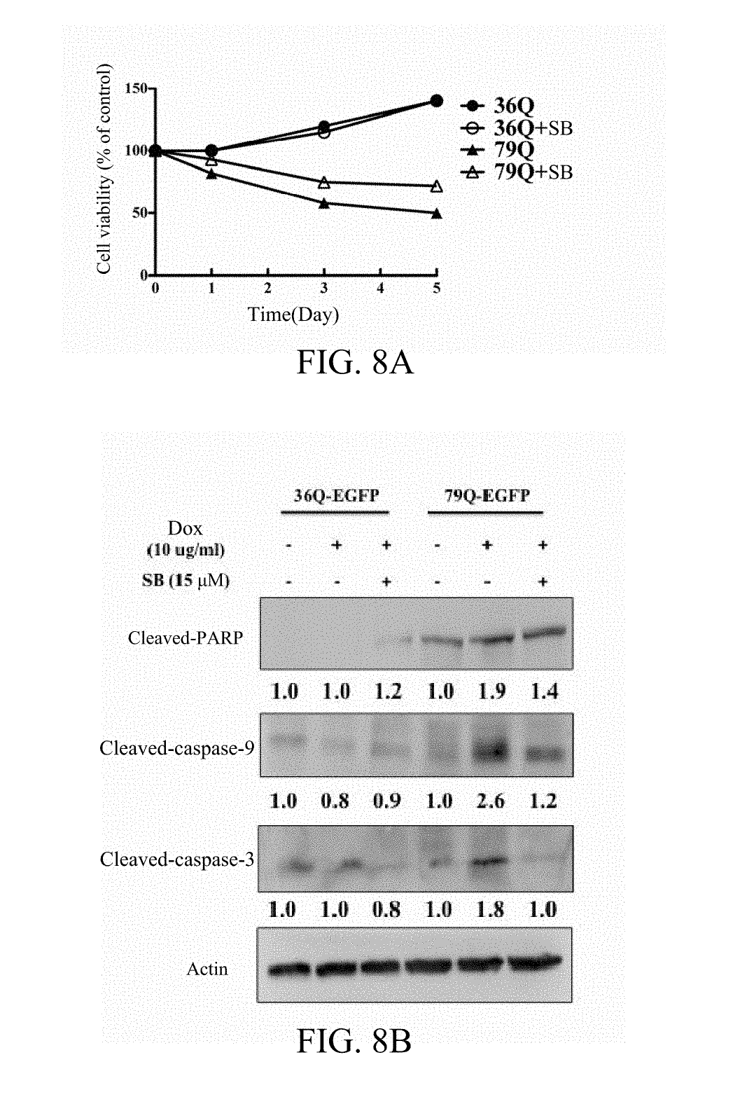

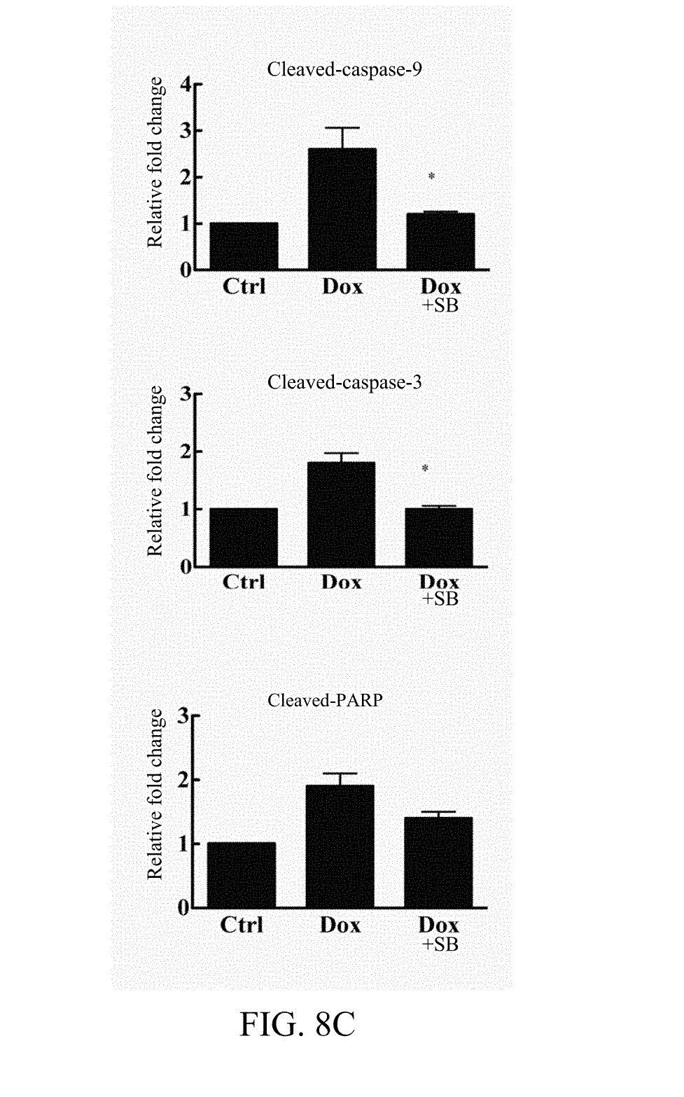

The cell viability was measured using MTT colorimetric assay as mentioned previously. The test results are shown in FIG. 8A. For nTBP/Q₃₆-EGFP cells, the cell viability did not change with or without the treatment of silibinin. For nTBP/Q₇₉-EGFP cells, the cell viability increased approximately 20% after the treatment of 15 μM silibinin for 5 days. It demonstrates that silibinin provides protection by inhibiting the cytotoxicity induced by nTBP/Q₇₉-EGFP.

Protein Expression of Caspase Family Induced by Dox in nTBP/Q₇₉-EGFP Cells

Western blotting was used to determine whether silibinin can inhibit cell death due to the expression of nTBP/Q₇₉-EGFP via caspase-dependent apoptosis pathway.

nTBP/Q₃₆-EGFP cells and nTBP/Q₇₉-EGFP cells were treated with 15 μM silibinin for 1 hour followed by the treatment of 10 μg/mL Dox and RA for 5 days.

The results were shown in FIGS. 8B and 8C. After the induced expression of nTBP/Q₇₉-EGFP for 5 days, the levels of cleaved-caspase-9, cleaved-caspase-3, and cleaved-PARP were up-regulated for 160%, 80% and 90%. However, after the treatment of 15 μM silibinin, the levels of cleaved-caspase-9, cleaved-caspase-3, and cleaved-PARP were inhibited respectively for about 88%, 100% and 44%. It indicates that silibinin inhibited the protein expression of caspase family induced by nTBP/Q₇₉-EGFP.

### Example 7

### The Effects of Silibinin on the Accumulation of Poly-Glutamine

In the present example, dot blot and western blotting methods were used to analyze whether silibinin can reduce the protein aggregation in nTBP/Q₇₉-EGFP cells induced by Dox.

nTBP/Q₃₆-EGFP cells and nTBP/Q₇₉-EGFP cells were cultured in 6 cm petri-dish for 24 hours followed by the treatment of 15 μM silibinin for 1 hour and the treatment of 10 μM Dox and 10 μM RA for 5 days. The proteins were extracted from the treated cells and analyzed by dot blot and western blotting, wherein the primary antibody is TBP(N-12) antibody and the secondary antibody is HRP conjugated anti-rabbit antibody.

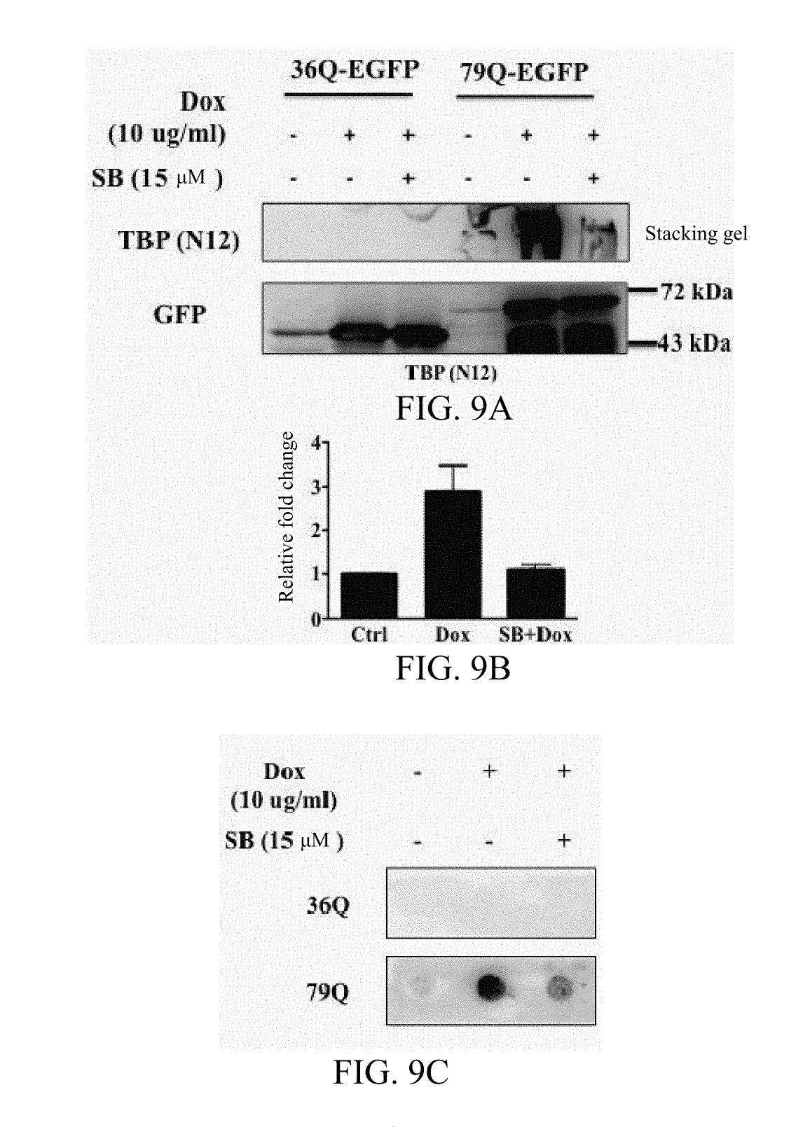

The results were shown in FIGS. 9A-9C. The results in both dot blot and western blotting showed that the protein aggregation in nTBP/Q₇₉-EGFP cells is higher than that in nTBP/Q₃₆-EGFP cells after the induction of 10 μM Dox for 5 days. However, after the treatment of 15 μM silibinin, the protein aggregation in nTBP/Q₇₉-EGFP cells were reduced 67% and 90% respectively in dot blot and western blotting. The results demonstrate that silibinin can increase cell viability by inhibiting nTBP/Q₇₉-EGFP protein aggregation.

The protein mis-folding and aggregation in brain are known to be the pathogenesis causes of several neuro-degenerative diseases. The pathogenesis of SCA17 is the TBP-N terminus protein aggregation which leads to the selective loss of neuron in cerebellum, especially the loss of Purkinje cells. The present disclosure proves that silibinin can effectively inhibit the formation of protein aggregation in nTBP/Q₇₉-EGFP cells.

### Example 8 Animal Studies

### The Effects of Silibinin on the Motor Behavior of SCA17 Transgenic Mice

In order to understand the efficacy of silibinin in treating SCA17 in vivo, SCA17 transgenic mice (referring as SCA17 mice hereinafter) were used, which were kindly provided by Dr. Hsiu-Mei Hsieh in NTNU.

The mice were housed individually in ventilated cages with a 12-hour light/dark cycle. All mice were maintained in the animal facility in NTNU under specific pathogen-free conditions in accordance with institutional guidelines of the Animal Care and Use Committee at NTNU. The experimental animals were used for motor behavioral assessments, footprint test and the protein aggregation analysis in cerebellum.

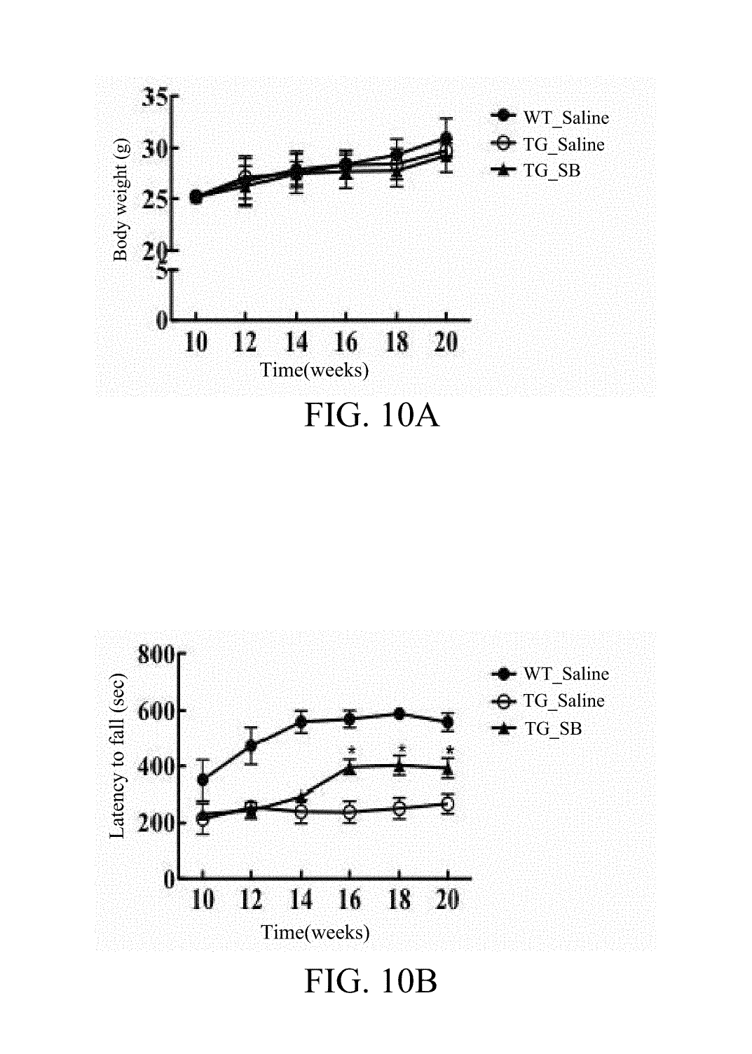

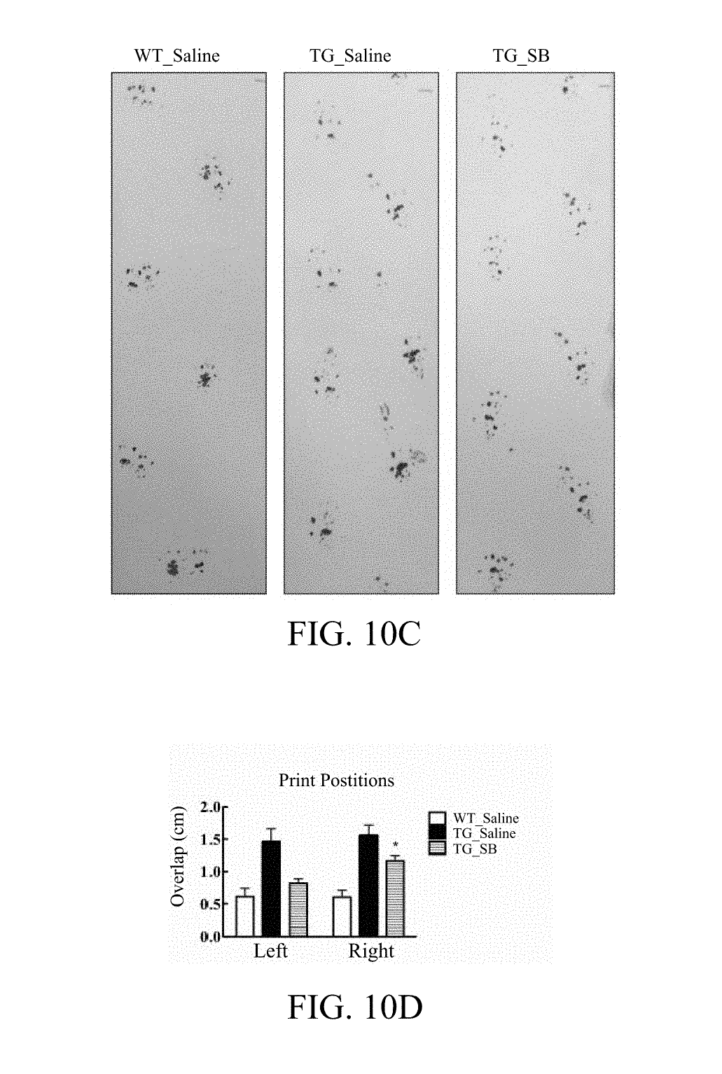

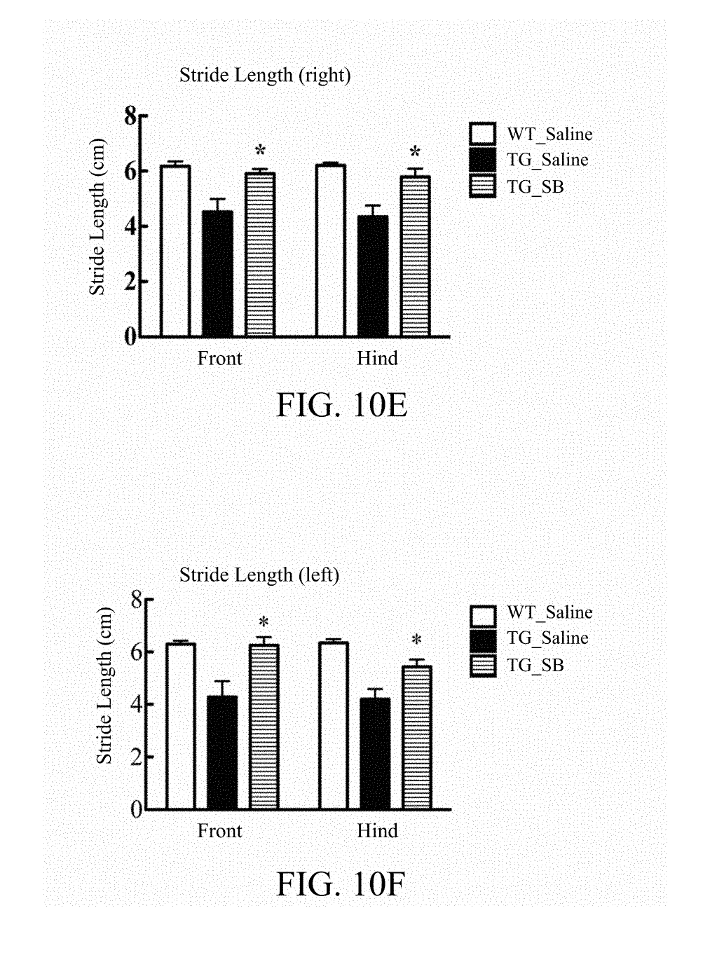

Since the SCA17 mice were 8 weeks old, the mice were injected intraperitoneally with silibinin in saline at 4.5 mg/kg once every 2 days, designated as TG_SB group. The mice in TG_saline group were injected with same amount of saline. As shown in FIG. 10A, the weights of the SCA17 transgenic mice which were 10 to 20 weeks old had no significant differences when compared to the control group (WT_saline group).

At the 10^{th} week, the rotarod test was performed to measure the motor coordination of the mice. The mice were trained prior to drug treatment. The mouse was placed on the rotarod with acceleration from 2 to 20 rpm for 5 minutes maintaining at 20 rpm for 5 more minutes to establish the baseline of the behavior of the mouse.

The condition was maintained at a linear acceleration from 4 to 30 rpm within 5 minutes during the testing period. The rotarod test was performed once every 2 weeks, since the mouse was 10 weeks old till 20 weeks old. Each test has 3 repeats with a maximum duration of no more than 600 seconds. The latency of fall was recorded. The test was conducted between 12:00 and 18:00.

The results of rotarod test were shown in FIG. 10B. For the SCA17 mouse injected with saline, the average time of the latency of fall is 269±36 seconds. The average time is 558±33 seconds for the control group. However, for the SCA17 mouse injected with silibinin, the average time of the latency of fall showed improvement as 393±36 seconds.

In addition, footprint pattern analysis was conducted to observe any abnormity of the gait of the mice. Such test was conducted widely to determine motor skill, coordination and balance. The hindfeet and forefeet of the mouse were coated with red and blue nontoxic paint respectively, and the mouse was allowed to walk along a runway on a fresh sheet of white paper. The distance between the centers of the hind footprint and the fore footprint, the length of the step, and the parallel distance were measured for a continuous 6 steps, excluding the steps in the beginning and at the end. The test was performed once every half month.

As shown in FIGS. 10C and 10D, for the SCA 17 mouse injected with saline, the overlapped lengths of left-paw and right-paw were 1.5±0.2 cm and 1.6±0.2 cm respectively, which were longer than those of the mouse in control group (which were 0.6±0.1 cm). However, for the SCA 17 mouse injected with silibinin, the overlapped lengths of left-paw and right-paw were 0.8±0.1 cm and 1.1±0.1 cm respectively. As shown in FIGS. 10E and 10F, the length of the steps of the SCA17 mouse injected with silibinin was comparable to that of the control group. From the above, the application of silibinin indeed improves the defect of SCA17 mouse in motor coordination.

### The Effects of Silibinin in the Levels of Poly-Glutamine Accumulation and Cleaved-Caspase-3 in the Cerebellum of SCA17 Mouse

In order to understand whether silibinin can reduce TBP/polyQ protein aggregation and the expression of cleaved-caspase 3 in the cerebellum of SCA17 mouse, TBP/polyQ protein aggregation and the expression of cleaved-caspase 3 in the cerebellum of SCA17 mouse were measured.

All SCA17 mice at 20 weeks old at the end of the experiments were anesthetized with urethane (1.5 mg/kg, intraperitoneal injection) for 10 minutes, the cerebellum of the mice were removed (6 samples per group), placed in cold RIPA buffer, and homogenized to measure the protein expression levels.

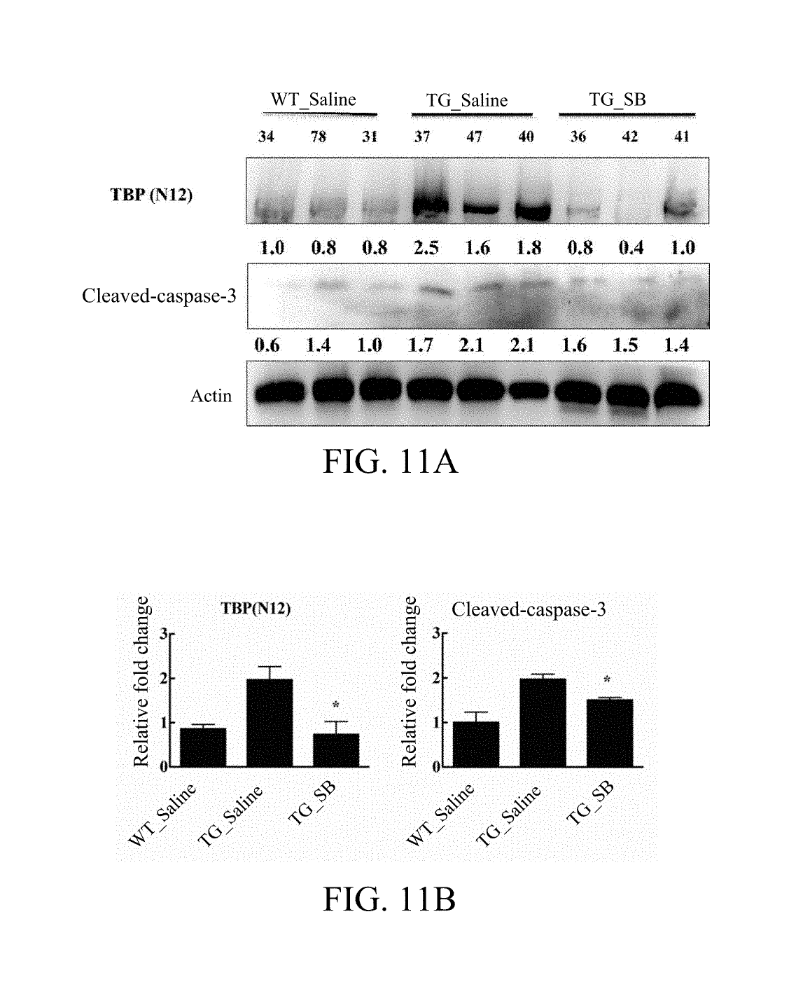

As shown in FIGS. 11A and 11B, the TBP/polyQ protein aggregation and the protein level of cleaved caspase 3 were both increased at 100% in the cerebellum of the SCA17 mice injected with saline in comparing to the mice in control group (WT_saline group). However, the TBP/polyQ protein aggregation and the protein level of cleaved caspase 3 were both inhibited at 100% and 25% in the cerebellum of the SCA17 mice injected with silibinin It demonstrates that the application of silibinin can effectively inhibit TBP/polyQ protein aggregation and the expression of cleaved caspase 3 in the cerebellum of the SCA17 mice.

The present examples demonstrate that silibinin can improve the motor behavior on rotarod and the abnormity of the gait in SCA17 mice, including shorter overlap of fore/hind foot prints and longer fore/hind step lengths. The TBP/polyQ protein aggregation and the level of cleaved caspase 3 in the cerebellum of the SCA17 mice were reduced due to the application of silibinin

Based on the above results, silibinin can reduce the pathogenesis cause of SCA17 by inhibiting mitochondria mediated apoptosis pathway and reducing TBP aggregation. In addition, it also demonstrated that silibinin can provide protective effects for the apoptosis induced by glutamate in SH-SY5Y and nTBP/Q₇₉-EGFP cells and can improve the motor behavior of the SCA17 mice. In summary, the method of silibinin application of the present disclosure can indeed prevent and treat SCA17 effectively.

The principles and effects of the present disclosure have been described using the above examples, which are not used to limit the present disclosure. Without departing from the spirit and scope of the present disclosure, any one skilled in the art can modify the above examples. Therefore, the scope of the present disclosure should be accorded with the claims appended.

The literatures cited by the present application are listed below, and each of the references is incorporated herein by reference.
1. Koeppen A H (2005) The pathogenesis of spinocerebellar ataxia. Cerebellum 4:62-73.
2. Carlson K M, Andresen J M, Orr H T (2009) Emerging pathogenic pathways in the spinocerebellar ataxias. Current opinion in genetics & development 19:247-253.
3. Paulson H (2009) The spinocerebellar ataxias. J Neuroophthalmol 29(3):227-237.
4. Orr H T and Zoghbi H Y (2007) Trinucleotide repeat disorders. Annual review of neuroscience 30:575-621.
5. Duenas A M, Goold R, Giunti P (2006) Molecular pathogenesis of spinocerebellar ataxias. Brain. 129:1357-1370.
6. Orr H T (2012) Cell biology of spinocerebellar ataxia. The Journal of cell biology 197:167-177.
7. Sakahira H, Breuer P, Hayer-Hard M K, Hartl F U (2002) Molecular chaperones as modulators of polyglutamine protein aggregation and toxicity. Proceedings of the National Academy of Sciences of the United States of America 99:16412-16418.
8. Popiel H A, Nagai Y, Onodera O, Inui T, Fujikake N, Urade Y, Strittmatter W J, Burke J R, Ichikawa A, Toda T (2004) Disruption of the toxic conformation of the expanded polyglutamine stretch leads to suppression of aggregate formation and cytotoxicity. Biochemical and biophysical research communications 317:1200-1206.
9. Margolis R L, O'Hearn E, Holmes S E, Srivastava A K, Mukherji M, Sinha K K (1993) Spinocerebellar Ataxia Type 12. Gene Reviews.
10. Matsuura T, Yamagata T, Burgess D L, Rasmussen A, Grewal R P, Watase K, Khajavi M, McCall A E, Davis C F, Zu L, Achari M, Pulst S M, Alonso E, Noebels J L, Nelson D L, Zoghbi H Y, Ashizawa T (2000) Large expansion of the ATTCT pentanucleotide repeat in spinocerebellar ataxia type 10. Nature genetics 26:191-194.
11. Mutsuddi M, Rebay I (2005) Molecular genetics of spinocerebellar ataxia type 8 (SCAB). RNA biology 2:49-52.
12. Chen D H, Cimino P J, Ranum L P, Zoghbi H Y, Yabe I, Schut L, Margolis R L, Lipe H P, Feleke A, Matsushita M, Wolff J, Morgan C, Lau D, Fernandez M, Sasaki H, Raskind W H, Bird T D (2005) The clinical and genetic spectrum of spinocerebellar ataxia 14. Neurology 64:1258-1260.
13. Misceo D, Fannemel M, Baroy T, Roberto R, Tvedt B, Jaeger T, Bryn V, Stromme P, Frengen E (2009) SCA27 caused by a chromosome translocation: further delineation of the phenotype. Neurogenetics 10:371-374.
14. Dick K A, Ikeda Y, Day J W, Ranum L P (2012) Spinocerebellar ataxia type 5.
Handbook of clinical neurology 103:451-459.
15. Gostout B, Liu Q, Sommer S S (1993) “Cryptic” repeating triplets of purines and pyrimidines (cRRY(i)) are frequent and polymorphic: analysis of coding cRRY(i) in the proopiomelanocortin (POMC) and TATA-binding protein (TBP) genes. American journal of human genetics 52:1182-1190.
16. Koide R, Kobayashi S, Shimohata T, Ikeuchi T, Maruyama M, Saito M, Yamada M, Takahashi H, Tsuji S (1999) A neurological disease caused by an expanded CAG trinucleotide repeat in the TATA-binding protein gene: a new polyglutamine disease? Human molecular genetics 8:2047-2053.
17. Nakamura K, Jeong S Y, Uchihara T, Anno M, Nagashima K, Nagashima T, Ikeda S, Tsuji S, Kanazawa I (2001) SCA17, a novel autosomal dominant cerebellar ataxia caused by an expanded polyglutamine in TATA-binding protein. Human molecular genetics 10:1441-1448.
18. Silveira I, Miranda C, Guimaraes L, Moreira M C, Alonso I, Mendonca P, Ferro A, Pinto-Basto J, Coelho J, Ferreirinha F, Poirier J, Parreira E, Vale J, Januario C, Barbot C, Tuna A, Barros J, Koide R, Tsuji S, Holmes S E, Margolis R L, Jardim L, Pandolfo M, Coutinho P, Sequeiros J (2002) Trinucleotide repeats in 202 families with ataxia: a small expanded (CAG)n allele at the SCA17 locus. Archives of neurology 59:623-629.
19. Reid S J, Rees M I, van Roon-Mom W M, Jones A L, MacDonald M E, Sutherland G, During M J, Faull R L, Owen M J, Dragunow M, Snell R G (2003) Molecular investigation of TBP allele length: a SCA17 cellular model and population study. Neurobiology of disease 13:37-45.
20. Rolfs A, Koeppen A H, Bauer I, Bauer P, Buhlmann S, Topka H, Schols L, Riess O (2003) Clinical features and neuropathology of autosomal dominant spinocerebellar ataxia (SCA17). Annals of neurology 54:367-375.
21. Kasumu A and Bezprozvanny (2012) Deranged calcium signaling in Purkinje cells and pathogenesis in spinocerebellar ataxia 2 (SCA2) and other ataxias. Cerebellum 11:630-639.
22. Duchen M R (2012) Mitochondria, calcium-dependent neuronal death and neurodegenerative disease. European journal of physiology 464:111-121.
23. Ishikawa K, Ishiguro T, Takahashi M, Sato N, Amino T, Niimi Y, Mizusawa H (2009) Molecular genetic approach to spinocerebellar ataxias. Clinical neurology 49:907-909.
24. Lee L C, Chen C M, Chen F L, Lin P Y, Hsiao Y C, Wang P R, Su M T, Hsieh-Li H M, Hwang J C, Wu C H, Lee G C, Singh S, Lin Y, Hsieh S Y, Lee-Chen G J, Lin J Y (2009) Altered expression of HSPA5, HSPA8 and PARK7 in spinocerebellar ataxia type 17 identified by 2-dimensional fluorescence difference in gel electrophoresis. ClinChimActa 400(1-2):56-62.
25. Matilla-Duenas A, Sanchez I, Corral-Juan M, Davalos A, Alvarez R, Latorre P (2010) Cellular and molecular pathways triggering neurodegeneration in the spinocerebellar ataxias. Cerebellum 9:148-166.
26. Kim S, Nollen E A, Kitagawa K, Bindokas V P, Morimoto R I (2002) Polyglutamine protein aggregates are dynamic Nature cell biology 4:826-831.
27. Seidel K, Siswanto S, Brunt E R, den Dunnen W, Korf H W, Rub U (2012) Brain pathology of spinocerebellar ataxias. Actaneuropathologica 124:1-21.
28. Friedman M J, Li S, Li X J (2009) Activation of gene transcription by heat shock protein 27 may contribute to its neuronal protection. The Journal of biological chemistry 284:27944-27951.
29. Uchihara T, Fujigasaki H, Koyano S, Nakamura A, Yagishita S, Iwabuchi K (2001) Non-expanded polyglutamine proteins in intranuclear inclusions of hereditary ataxias—triple-labeling immunofluorescence study. Actaneuropathologica 102:149-152.
30. Ghosh T, Pandey N, Maitra A, Brahmachari S K, Pillai B (2007) A role for voltage-dependent anion channel Vdac1 in polyglutamine-mediated neuronal cell death. PloS one 2:e1170.
31. Roshan R, Ghosh T, Gadgil M, Pillai B (2012) Regulation of BACE1 by miR-29a/b in a cellular model of Spinocerebellar Ataxia 17. RNA biology 9:891-899.
32. Mattson M P (2008) Glutamate and neurotrophic factors in neuronal plasticity and disease. Annals of the New York Academy of Sciences 1144:97-112.
33. Lau A and Tymianski M (2010) Glutamate receptors, neurotoxicity and neurodegeneration. Pflugers Archiv: European journal of physiology 460:525-542.
34. Yang J L, Sykora P, Wilson D M, Mattson M P, Bohr V A (2011) The excitatory neurotransmitter glutamate stimulates DNA repair to increase neuronal resiliency. Mechanisms of ageing and development 132:405-411.
35. Vaarmann A, Kovac S, Holmstrom K M, Gandhi S, Abramov A Y (2013) Dopamine protects neurons against glutamate-induced excitotoxicity. Cell Death Dis 4 e455.
36. Arundine M, Tymianski M (2004) Molecular mechanisms of glutamate-dependent neurodegeneration in ischemia and traumatic brain injury. Cellular and molecular life sciences: Cellular and Molecular Life Sciences 61:657-668.
37. Sun Z W, Zhang L, Zhu S J, Chen W C, Mei B (2010) Excitotoxicity effects of glutamate on human neuroblastoma SH-SY5Y cells via oxidative damage. Neuroscience bulletin 26:8-16.
38. Hirashima Y, Kurimoto M, Nogami K, Endo S, Saitoh M, Ohtani O, Nagata T, Muraguchi A, Takaku A (1999) Correlation of glutamate-induced apoptosis with caspase activities in cultured rat cerebral cortical neurons. Brain Res 849:109-118.
39. Delgado-Rubin de Celix A, Chowen J A, Argente J, Frago L M (2006) Growth hormone releasing peptide-6 acts as a survival factor in glutamate-induced excitotoxicity. Journal of neurochemistry 99:839-849.
40. Di X, Yan J, Zhao Y, Zhang J, Shi Z, Chang Y, Zhao B (2010) L-theanine protects the APP (Swedish mutation) transgenic SH-SY5Y cell against glutamate-induced excitotoxicity via inhibition of the NMDA receptor pathway. Neuroscience 168:778-786.
41. Anderton R S, Meloni B P, Mastaglia F L, Greene W K, Boulos S (2011) Survival of motor neuron protein over-expression prevents calpain-mediated cleavage and activation of procaspase-3 in differentiated human SH-SY5Y cells. Neuroscience 181:226-233.
42. Miao Y, Dong L D, Chen J, Hu X C, Yang X L, Wang Z (2012) Involvement of calpain/p35-p25/Cdk5/NMDAR signaling pathway in glutamate-induced neurotoxicity in cultured rat retinal neurons. PloS one 7:e42318.
43. Kristal B S and Dubinsky J M (1997) Mitochondrial permeability transition in the central nervous system: induction by calcium cycling-dependent and -independent pathways. Journal of neurochemistry 69:524-538.
44. Kroemer G, Dallaporta B, Resche-Rigon M (1998) The mitochondrial death/life regulator in apoptosis and necrosis. Annual review of physiology 60:619-642.
45. McStay G P, Clarke S J, Halestrap A P (2002) Role of critical thiol groups on the matrix surface of the adenine nucleotide translocase in the mechanism of the mitochondrial permeability transition pore. The Biochemical journal 367:541-548.
46. Votyakova T V and Reynolds I J (2005) Ca²⁺-induced permeabilization promotes free radical release from rat brain mitochondria with partially inhibited complex I. Journal of neurochemistry 93:526-537.
47. Clausen A, McClanahan T, Ji S G, Weiss J H (2013) Mechanisms of rapid reactive oxygen species generation in response to cytosolic Ca²⁺ or Zn²⁺ loads in cortical neurons. PloS one 8:e83347.
48. Lai T W, Zhang S, Wang Y T (2013) Excitotoxicity and stroke: Identifying novel targets for neuroprotection. Progress in neurobiology 115:157-88.

What is claimed is:

1. A method for preventing or treating spinocerebellar ataxia by administrating a pharmaceutical composition comprising an effective amount of silibinin to a subject in need thereof.

2. The method of claim 1, wherein the spinocerebellar ataxia is associated with aberrant accumulation of poly-glutamine proteins.

3. The method of claim 2, wherein the spinocerebellar ataxia is spinocerebellar ataxia type 1 (SCA1), spinocerebellar ataxia type 2 (SCA2), spinocerebellar ataxia type 3 (SCA3), spinocerebellar ataxia type 6 (SCA6), spinocerebellar ataxia type 7 (SCA7), or spinocerebellar ataxia type 17 (SCA17).

4. The method of claim 2, wherein the pharmaceutical composition reduces the aberrant accumulation of poly-glutamine proteins in the subject.

5. The method of claim 1, wherein a concentration of the silibinin in the pharmaceutical composition is in a range from 5 μM to 30 μM.

6. The method of claim 1, wherein the effective amount of the silibinin is in a range from 0.125 mg to 9 mg per kilogram of a body weight of the subject.

7. The method of claim 1, wherein the pharmaceutical composition is administrated to the subject through an injection.

8. A method for preventing or treating neuron damage by administrating a pharmaceutical composition comprising an effective amount of silibinin to a subject in need thereof.

9. The method of claim 8, wherein the neuron damage is caused by glutamatergic excitotoxicity, apoptosis induced by glutamate, or apoptosis mediated by mitochondria.

10. The method of claim 8, wherein the pharmaceutical composition inhibits an apoptosis pathway.

11. The method of claim 10, wherein the apoptosis pathway is a calcium dependent apoptosis pathway, a mitochondria dependent apoptosis pathway or a caspase dependent apoptosis pathway.

12. The method of claim 10, wherein the pharmaceutical composition inhibits an expression level of at least one of calpain-2 and calpain-specific a-spectrin breakdown product (SBDP) to inhibit the apoptosis pathway.

13. The method of claim 10, wherein the pharmaceutical composition inhibits an expression level of Bax and/or increases an expression level of Bcl-2 to inhibit the apoptosis pathway.

14. The method of claim 10, wherein the pharmaceutical composition inhibits at least one of cleaved caspase-9, cleaved caspase-3, and cleaved poly(ADP-ribose) polymerase (PARP) to inhibit the apoptosis pathway.

15. The method of claim 8, wherein the pharmaceutical composition reduces generation of cytosolic reactive oxygen species induced by glutamate.

16. The method of claim 8, wherein the pharmaceutical composition reduces loss of mitochondria potential induced by glutamate.

17. The method of claim 8, wherein the pharmaceutical composition reduces protein aggregation in a neuron.

18. The method of claim 8, wherein a concentration of the silibinin in the pharmaceutical composition is in a range from 5 μM to 30 μM.

19. A method for preventing or treating neuron damage associated with aberrant accumulation of poly-glutamine proteins by administrating a pharmaceutical composition comprising an effective amount of silibinin to a subject in need thereof, wherein the effective amount of the silibinin is in a range from 0.125 mg to 9 mg per kilogram of a body weight of the subject.

20. The method of claim 19, wherein the neuron damage associated with the aberrant accumulation of poly-glutamine proteins is spinocerebellar ataxia type 17 (SCA17).
